(19) United States
(12) Patent Application Publication
(10) Pub. No.: US 2024/0188937 A1
(43) Pub. Date: Jun. 13, 2024

(54) ULTRASOUND DIAGNOSTIC APPARATUS

(71) Applicant: FUJIFILM Healthcare Corporation, Chiba (JP)

(72) Inventor: AYA SODEYAMA, Tokyo (JP)

(73) Assignee: FUJIFILM Healthcare Corporation, Chiba (JP)

(21) Appl. No.: 18/534,696

(22) Filed: Dec. 10, 2023

(30) Foreign Application Priority Data
Dec. 13, 2022 (JP) 2022-198829

(51) Int. Cl. A61B 8/08; G01S 7/52; G06T 5/00; G06T 7/00

(52) U.S. Cl. CPC A61B 8/5269; A61B 8/5207; G01S 7/52077; G06T 5/002; G06T 5/008; G06T 7/0012; G06T 2207/10132; G06T 2207/30168

(57) ABSTRACT

A characteristic estimation unit analyzes a reception signal before detection processing among reception signals obtained by transmitting and receiving ultrasound waves with respect to a subject, to estimate a signal characteristic of the reception signal for each region in a data space of the reception signal. An image processing parameter decision unit decides an image processing parameter for enhancing an image quality of an ultrasound image based on the signal characteristic, for each region in the data space of the reception signal, which is estimated by the characteristic estimation unit. An image formation unit executes image quality enhancement processing of the ultrasound image based on the image processing parameter decided by the image processing parameter decision unit while forming the ultrasound image based on the reception signal after the detection processing.

### CROSS-REFERENCE TO RELATED APPLICATION

This application claims the priority benefit of Japan application serial no. 2022-198829, filed on Dec. 13, 2022. The entirety of the above-mentioned patent application is hereby incorporated by reference herein and made a part of this specification.

### BACKGROUND

### 1. Technical Field

The present disclosure relates to an improvement of an ultrasound diagnostic apparatus.

### 2. Description of the Related Art

In the related art, an ultrasound diagnostic apparatus has been known, which transmits and receives ultrasound waves to and from a subject, forms an ultrasound image based on a reception signal obtained by transmitting and receiving the ultrasound waves, and displays the formed ultrasound image on a display. In the related art, various techniques for enhancing an image quality (for example, noise reduction, brightness correction, or the like) of the ultrasound image formed by the ultrasound diagnostic apparatus have been proposed.

For example, JP2011-251136A or JP2008-278995A discloses an ultrasound diagnostic apparatus that forms an ultrasound image based on a reception signal received by an ultrasound probe, performs multi-resolution analysis with respect to the formed ultrasound image to calculate an expansion coefficient (decomposition coefficient) for each position and for each resolution, estimates a noise amount based on the obtained expansion coefficient, and applies a gain according to the estimated noise amount to each expansion coefficient to reduce noise of the ultrasound image.

Further, JP2020-508168A or JP2004-500915A discloses an ultrasound diagnostic apparatus that specifies a noise signal based on a reception signal output by a reception circuit in an environment in which an oscillation element array does not receive reflected waves from a subject (for example, in a case in which ultrasound waves are transmitted toward the air), executes noise reduction processing with respect to a reception signal based on the reflected waves from the subject based on the specified the noise signal, and forms an ultrasound image based on the reception signal in which the noise is reduced.

### SUMMARY OF THE INVENTION

For example, as in JP2011-251136A or JP2008-278995A, in the related art, an image processing parameter, which is a parameter used for formation processing or correction processing of the ultrasound image, is decided for each region of the ultrasound image by analyzing the ultrasound image, and processing of enhancing an image quality of the ultrasound image for each region is performed based on the decided image processing parameter. However, in the related art, data that is an analysis target for deciding the image processing parameter is exclusively the reception signal after detection processing of the ultrasound image or the like.

The detection processing reduces an amount of information in the reception signal. For example, phase information of each reception signal is lost due to the detection processing. Therefore, in deciding the image processing parameter for enhancing an image quality for each region of the ultrasound image, in a case in which the data that is the analysis target is the reception signal after the detection processing, there is a limit in the analysis, and a suitable image processing parameter cannot be decided in some cases. Stated another way, by setting the data that is the analysis target for deciding the image processing parameter as the reception signal before the detection processing, it is expected that a more suitable image processing parameter can be decided, and as a result, the image quality of the ultrasound image can be more suitably enhanced.

It should be noted that, in the related art, as in JP2020-508168A or JP2004-500915A, for example, processing of the reception signal itself is performed based on the analysis result of the reception signal before the detection processing. However, in the related art, the decision of the image processing parameter for enhancing an image quality of the ultrasound image based on the analysis result of the reception signal before the detection processing has not been performed.

An object of an ultrasound diagnostic apparatus of the present disclosure is to enable enhancement of an image quality of an ultrasound image based on an image processing parameter decided based on a signal characteristic of a reception signal before detection processing.

An aspect of the present disclosure relates to an ultrasound diagnostic apparatus comprising: a characteristic estimation unit that analyzes a reception signal before detection processing among reception signals obtained by transmitting and receiving ultrasound waves with respect to a subject, to estimate a signal characteristic of the reception signal for each region in a data space of the reception signal; an image processing parameter decision unit that decides an image processing parameter based on the estimated signal characteristic for each region; and an image formation unit that forms an ultrasound image subjected to image quality enhancement processing based on the image processing parameter based on the reception signal subjected to the detection processing.

In addition, the signal characteristic may be at least one of coherence of each reception signal output by each oscillation element that transmits and receives the ultrasound waves, an S/N ratio calculated by frequency analysis with respect to the reception signal, or attenuation information indicating attenuation of signal intensity in a depth direction of the subject, which is calculated by the frequency analysis with respect to the reception signal, the attenuation information being estimated by excluding the signal intensity of the region which includes a structure in the subject and is specified based on the reception signal before the detection processing.

With this configuration, the signal characteristic is estimated for each region of the data space of the reception signal based on at least the reception signal before the detection processing, which has an abundant amount of information than the reception signal after the detection processing, and the image quality enhancement processing of the ultrasound image is executed by the image processing parameter decided based on the signal characteristic. As a result, more suitable image quality enhancement processing (processing of realizing lower noise or more suitable time gain control (TGC)) can be performed.

The image processing parameter may be a brightness transformation coefficient for transforming brightness of a pixel of the ultrasound image based on at least one of the coherence or the S/N ratio related to the region, and the brightness transformation coefficient may be a coefficient for making brightness of a pixel included in the region larger as at least one of the coherence or the S/N ratio related to the region is larger.

As less noise is included in the reception signal, the coherence of the reception signal is larger. It is needless to say that the S/N ratio of the reception signal is larger as the less noise is included in the reception signal. With this configuration, by transforming the brightness of the ultrasound image by the brightness transformation coefficient decided based on at least one of the coherence or the S/N ratio before the detection processing, the brightness of the region including a large amount of the signal components is further increased, and the brightness of the region including a large amount of the noise components is further decreased. In other words, the noise of the ultrasound image is reduced.

The image formation unit may perform multi-resolution analysis with respect to the ultrasound image formed based on the reception signal subjected to the detection processing and executes noise reduction of the ultrasound image by applying a gain according to magnitude of an absolute value of each obtained expansion coefficient to each expansion coefficient, and the image processing parameter may be the gain corrected to be larger as at least one of the coherence or the S/N ratio related to the region is larger.

With this configuration, in the multi-resolution analysis, the gain applied to the expansion coefficient including more noise is reduced, so that the noise of the ultrasound image is reduced.

The image processing parameter may be a brightness correction function for time gain control based on the attenuation information, and the image formation unit may execute the time gain control with respect to the region of the ultrasound image including the structure based on the brightness correction function.

With this configuration, even in a case in which there is data corresponding to the structure of the subject in the data space of the reception signal, the brightness correction function that is not affected by the structure can be decided, and as a result, more suitable TGC can be performed.

With the ultrasound diagnostic apparatus disclosed in the present disclosure, it is possible to enhance the image quality of the ultrasound image based on the image processing parameter decided based on the signal characteristic of the reception signal before the detection processing.

### BRIEF DESCRIPTION OF THE DRAWINGS

FIG. 1 is a schematic configuration diagram of an ultrasound diagnostic apparatus according to the present embodiment.

FIG. 2 is a conceptual diagram showing an example of a reception signal after delay processing.

FIG. 3A is a diagram showing a power spectrum obtained by frequency analysis of an amplitude value distribution of a reception signal group of A of FIG. 2.

FIG. 3B is a diagram showing a power spectrum obtained by frequency analysis of an amplitude value distribution of a reception signal group of B of FIG. 2.

FIG. 3C is a diagram showing a power spectrum obtained by frequency analysis of an amplitude value distribution of a reception signal group of C of FIG. 2.

FIG. 4 is a conceptual diagram showing the reception beam data and a region of the reception beam data in a data space.

FIG. 5A is a first diagram showing an example of a γ curve used in brightness transformation processing.

FIG. 5B is a second diagram showing an example of the γ curve used in the brightness transformation processing.

FIG. 6 is an explanatory diagram showing a flow of processing of multi-resolution analysis.

FIG. 7A is a diagram showing an example of an expansion coefficient transformation function in the related art.

FIG. 7B is a diagram showing an example of an expansion coefficient transformation function in the present embodiment.

FIG. 8 is a flowchart showing a flow of processing of the ultrasound diagnostic apparatus according to the present embodiment.

### DESCRIPTION OF THE EMBODIMENTS

### Schematic Configuration of Ultrasound Diagnostic Apparatus

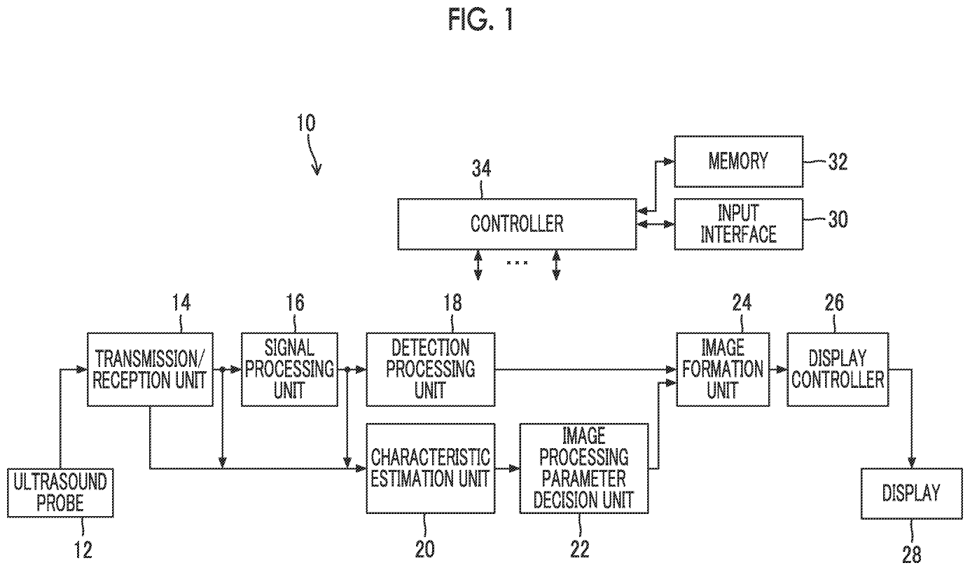

FIG. 1 is a schematic configuration diagram of an ultrasound diagnostic apparatus 10 according to the present embodiment. The ultrasound diagnostic apparatus 10 is a medical apparatus that is installed in a medical institution, such as a hospital, and is used during an ultrasound examination.

The ultrasound diagnostic apparatus 10 is an apparatus that scans a subject with an ultrasound beam to generate an ultrasound image based on a reception signal obtained by the scanning. For example, the ultrasound diagnostic apparatus 10 forms a tomographic image (B-mode image) in which the amplitude intensity of reflected waves from a scanning surface is transformed into the brightness based on the reception signal. Alternatively, the ultrasound diagnostic apparatus 10 can also form a Doppler image, which is an ultrasound image showing a motion velocity of a tissue in the subject, based on a difference (Doppler shift) between frequencies of transmitted waves and received waves. In the present embodiment, processing of generating the B-mode image by the ultrasound diagnostic apparatus 10 will be described.

An ultrasound probe 12 is a device that transmits and receives ultrasound waves to and from the subject. The ultrasound probe 12 has an oscillation element array including a plurality of oscillation elements that transmit and receive the ultrasound waves to and from the subject.

A transmission/reception unit 14 transmits a transmission signal to the ultrasound probe 12 (specifically, each oscillation element of the oscillation element array) under the control of a controller 34 (described later). As a result, the ultrasound waves are transmitted from each oscillation element toward the subject. In addition, the transmission/reception unit 14 receives a reception signal from each oscillation element that receives the reflected waves from the subject. The transmission/reception unit 14 includes an adder and a plurality of delayers corresponding to the respective oscillation elements, and phase adjustment addition processing of aligning and adding phases of the reception signals from the respective oscillation elements is performed by the adder and the plurality of delayers. As a result, reception beam data in which information indicating the signal intensity of the reflected waves from the subject is arranged in a depth direction of the subject is formed. Processing of forming the reception beam data is referred to as reception beam forming.

The signal processing unit 16 executes various types of signal processing including, for example, filter processing of applying a bandpass filter to the reception beam data from the transmission/reception unit 14.

At least one of the reception signal before the reception beam forming by the transmission/reception unit 14, the reception signal (reception beam data) after the reception beam forming by the transmission/reception unit 14, or the reception signal after the filter processing by the signal processing unit 16 is transmitted to a characteristic estimation unit 20 described later.

A detection processing unit 18 executes processing, such as detection processing (for example, envelope detection processing) or logarithmic compression processing, with respect to the reception signal after the processing by the signal processing unit 16. The reception signal loses the phase information (frequency information) due to the detection processing by the detection processing unit 18. That is, an amount of information of the reception signal after the detection processing is smaller than an amount of information of the reception signal before the detection processing.

The characteristic estimation unit 20 analyzes the reception signal before the detection processing among the reception signals obtained by transmitting and receiving the ultrasound waves to the subject. The reception signal before the detection processing is, for example, the reception signal before the reception beam forming by the transmission/reception unit 14, the reception signal (reception beam data) after the reception beam forming by the transmission/reception unit 14, or the reception signal after the various types of signal processing including the filter processing by the signal processing unit 16. As a result, the characteristic estimation unit 20 estimates the signal characteristic of the reception signal for each region in the data space of the reception signal (reception beam data) corresponding to the region of the ultrasound image formed later. Details of the processing of the characteristic estimation unit 20 will be described later.

An image processing parameter decision unit 22 decides an image processing parameter based on the signal characteristic, for each region in the data space of the reception beam data, which is estimated by the characteristic estimation unit 20. It should be noted that the image processing parameter means a parameter for enhancing an image quality of the ultrasound image, the parameter being used in formation processing or correction processing of the ultrasound image. Details of the processing of the image processing parameter decision unit 22 will be described later.

An image formation unit 24 forms the ultrasound image (B-mode image) based on the reception signal subjected to the detection processing or the like by the detection processing unit 18. In particular, the image formation unit 24 executes image quality enhancement processing of the ultrasound image based on the image processing parameter decided by the image processing parameter decision unit 22. The image quality enhancement processing of the ultrasound image by the image formation unit 24 will be described later.

A display controller 26 performs control of displaying, on a display 28, the ultrasound image formed by the image formation unit 24 and various types of other information. The display 28 is, for example, a display device configured of a liquid crystal display, an organic electro luminescence (EL), or the like.

An input interface 30 is configured of, for example, a button, a track ball, a touch panel, or the like. The input interface 30 is used to input a command from a user to the ultrasound diagnostic apparatus 10.

A memory 32 includes a hard disk drive (HDD), a solid state drive (SSD), an embedded Multimedia card (eMMC), a read only memory (ROM), or the like. The memory 32 stores an ultrasound diagnostic program for operating each of the units of the ultrasound diagnostic apparatus 10. It should be noted that the ultrasound diagnostic program can also be stored, for example, in a computer-readable non-transitory storage medium, such as a universal serial bus (USB) memory or a CD-ROM. The ultrasound diagnostic apparatus 10 can read and execute the ultrasound diagnostic program from such a storage medium.

The controller 34 includes at least one of a general-purpose processor (for example, a central processing unit (CPU)) or a dedicated processor (for example, a graphics processing unit (GPU), an application specific integrated circuit (ASIC), a field-programmable gate array (FPGA), a programmable logic device, and the like). The controller 34 may be configured by the cooperation of a plurality of processing devices that are present at physically separated positions, instead of being configured of one processing device. The controller 34 controls each of the units of the ultrasound diagnostic apparatus 10 according to the ultrasound diagnostic program stored in the memory 32.

It should be noted that each of the units of the transmission/reception unit 14, the signal processing unit 16, the detection processing unit 18, the characteristic estimation unit 20, the image processing parameter decision unit 22, the image formation unit 24, and the display controller 26 is configured of one or a plurality of processors, chips, electric circuits, or the like. Each of these units may be realized by the cooperation between hardware and software.

The schematic configuration of the ultrasound diagnostic apparatus 10 is described above. Hereinafter, details of the image quality enhancement processing based on the image processing parameter by the characteristic estimation unit 20, the image processing parameter decision unit 22, and the image formation unit 24 will be described.

### Estimation of Signal Characteristic

In the present embodiment, the characteristic estimation unit 20 estimates, as the signal characteristic of the reception signals, at least one of coherence of individual reception signal (referred to as “individual reception signal” in the present disclosure) output by each oscillation element that transmits and receives the ultrasound waves, an S/N ratio calculated based on the signal intensity acquired from the reception signal, or attenuation information indicating attenuation of the signal intensity of the reception signal in the depth direction of the subject. Each estimation method will be described below. It should be noted that the signal characteristic of the reception signal estimated by the characteristic estimation unit 20 is not limited to these.

### Estimation Method of Coherence

First, the estimation method of the coherence of each individual reception signal will be described. The coherence means a degree of phase alignment (after delay processing) of each individual reception signal. The characteristic estimation unit 20 estimates the coherence of each individual reception signal based on the reception signal (that is, the individual reception signal) before addition processing by the transmission/reception unit 14.

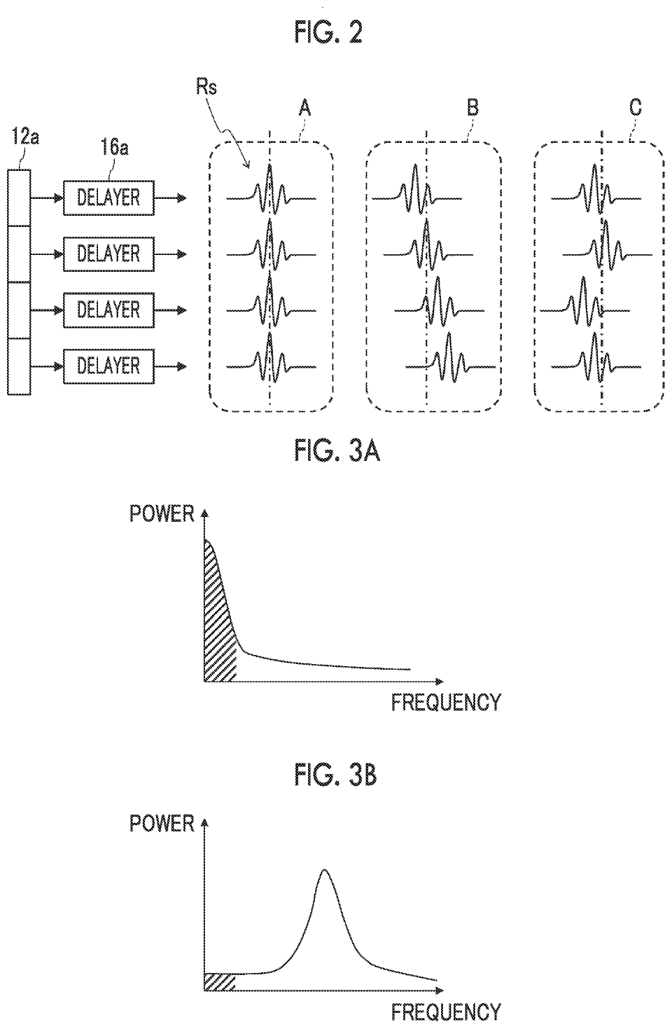

FIG. 2 schematically shows a plurality of oscillation elements 12a included in the oscillation element array of the ultrasound probe 12, a plurality of delayers 16a corresponding to the respective oscillation elements 12a included in the transmission/reception unit 14, and examples of sets (set A, set B, and set C) of the individual reception signals Rs after the delay processing by the plurality of delayers 16a.

The set A of the individual reception signals Rs is a set of the individual reception signals Rs in a case in which noise is not included so much. The individual reception signals Rs of the set A are mainly generated based on a main lobe component (reflected waves of the ultrasound waves transmitted substantially vertically from an ultrasound wave transmission surface of each oscillation element), and in this case, the phases of the respective individual reception signals Rs subjected to the delay processing by the delayer 16a are aligned. In other words, in this case, it can be said that the coherence of each individual reception signal Rs is large.

The set B and the set C of the individual reception signals Rs are sets of the individual reception signals Rs in a case in which the noise is included. The individual reception signals Rs of the set B are signals mainly including a side lobe component (reflected waves of the ultrasound waves transmitted obliquely from the ultrasound wave transmission surface of each oscillation element), and in this case, the phases of the respective individual reception signals Rs subjected to the delay processing by the delayer 16a are sequentially shifted in an arrangement direction of the oscillation elements 12a. In other words, in this case, it can be said that the coherence of each individual reception signal Rs is small. In addition, the individual reception signals Rs of the set C are signals in a case in which variation in sound velocity (phase aberration) occurs in the subject, and in this case, the phases of the respective individual reception signals Rs subjected to the delay processing by the delayer 16a are disturbed in pieces. In other words, in this case as well, it can be said that the coherence of each individual reception signal Rs is small.

As described above, a relevance is recognized between the noise included in the individual reception signal Rs and the coherence of the individual reception signal Rs from each oscillation element 12a. Specifically, as the less noise is included in the individual reception signal Rs, the coherence of each individual reception signal Rs is larger. Stated another way, as the more noise is included in the individual reception signal Rs, the coherence of each individual reception signal Rs is smaller.

The characteristic estimation unit 20 can estimate the coherence of each individual reception signal Rs by a method, such as a generalized coherence factor (GCF), a sign coherence factor (SCF), or a dual apodization with cross-correlation (DAX) method. The GCF, the SCF, and the DAX method are known techniques, but each of the GCF, the SCF, and the DAX method will be briefly described below.

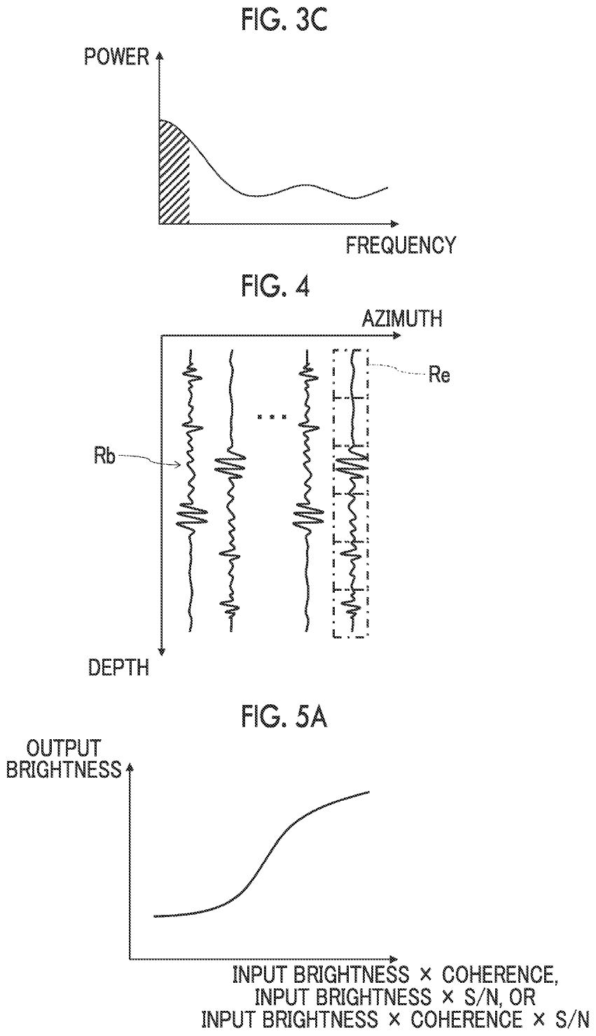

In the GCF, first, the characteristic estimation unit 20 acquires an amplitude value of each individual reception signal Rs output by each oscillation element 12a after the delay processing at a certain time point. As a result, an amplitude value distribution including a plurality of instantaneous amplitude values corresponding to the plurality of oscillation elements 12a is formed. The amplitude value distribution is formed at each time point (sampling timing), and the amplitude value distribution is changed dynamically. The characteristic estimation unit 20 performs a Fourier transform with respect to the amplitude value distribution. As a result, as shown in FIGS. 3A to 3C, it is possible to obtain a power spectrum in which a horizontal axis represents a frequency and a vertical axis represents a power. FIG. 3A is a power spectrum corresponding to the set A of the individual reception signals Rs of FIG. 2, FIG. 3B is a power spectrum corresponding to the set B of the individual reception signals Rs of FIG. 2, and FIG. 3C is a power spectrum corresponding to the set C of the individual reception signals Rs of FIG. 2.

In the set A of the individual reception signals Rs, since the phases of the respective individual reception signals Rs are aligned, a difference between the amplitude values in the amplitude value distribution is considerably small. Therefore, in the power spectrum of FIG. 3A corresponding to the set A of the individual reception signals Rs, a protruding peak appears in a DC region (diagonal line portion of FIG. 3A) which is a frequency region in the vicinity of the frequency 0.

In the set B of the individual reception signals Rs, since the phases of the respective individual reception signals Rs are sequentially shifted in the arrangement direction of the oscillation elements 12a, in the power spectrum of FIG. 3B corresponding to the set B of the individual reception signals Rs, a peak appears at a frequency determined according to an arrival direction (angle) of the side lobe component. In the power spectrum of FIG. 3B, the power of the DC region (diagonal line portion of FIG. 3B) is considerably small.

In the set C of the individual reception signals Rs, since the phases of the respective individual reception signals Rs are disturbed in pieces, the respective amplitude values in the amplitude value distribution also considerably vary. Therefore, in the power spectrum of FIG. 3C corresponding to the set C of the individual reception signals Rs, the power appears in a wide range of the frequency component. At least, in the power spectrum of FIG. 3C, the power of the DC region (diagonal line portion of FIG. 3C) does not protrude.

As described above, a relevance is recognized between the power of the DC region in the power spectrum obtained by performing the Fourier transform with respect to the amplitude value distribution and the coherence of each individual reception signal Rs. Therefore, the characteristic estimation unit 20 can obtain the coherence based on the power of the DC region in the power spectrum. Specifically, the characteristic estimation unit 20 calculates the coherence (here, GCF) of each individual reception signal Rs by Expression (1). The GCF as the coherence is larger as the less noise is included in the individual reception signal Rs, and is smaller as the more noise is included in the individual reception signal Rs.

GCF=(power of DC region)/(power of entire power spectrum) (1)

Next, in the SCF, first, the characteristic estimation unit 20 acquires the amplitude value of each individual reception signal Rs output by each oscillation element 12a after the delay processing at a certain time point to form the amplitude value distribution. Then, the individual reception signals Rs are binarized by Expression (2).

$$\begin{matrix}{b}_{i} & = - 1 if {R}_{s} < 0 \\ & = + 1 if {R}_{s} \geq 0\end{matrix} \qquad ( 2 )$$

In Expression (2), i is a channel number corresponding to each oscillation element 12a. In other words, the characteristic estimation unit 20 normalizes each individual reception signal Rs to a positive value of +1 and a negative value of −1.

Then, the characteristic estimation unit 20 calculates the SCF represented by Expression (3) as the coherence of each individual reception signal Rs.

$$SCF = {\vert 1 - \sqrt{1 - {\{ \frac{1}{N} {\sum }_{i = 0}^{N - 1} {b}_{i} \}}^{2}} \vert}^{p} \qquad ( 3 )$$

In Expression (3), N represents the number of the oscillation elements 12a, and p is a parameter for adjusting the SCF.

In a case in which the phases of the respective individual reception signals Rs are aligned as in the set A of the individual reception signals Rs, the values of bᵢ (i=0 to (N−1)) should be all +1 or all −1. In this case, the SCF is set to 1 according to Expression (3). On the other hand, in a case in which the phases of the respective individual reception signals Rs are not aligned as in the set B or the set C of the individual reception signals Rs, the values of bᵢ (i=0 to (N−1)) vary (are +1 or −1). In this case, as the phases of the respective individual reception signals Rs are disturbed, the SCF approaches 0. In other words, the SCF as the coherence is larger as the less noise is included in the individual reception signal Rs, and is smaller as the more noise is included in the individual reception signal Rs.

Finally, the DAX method is a method of performing the delay processing (that is, weighted delay processing) with respect to each individual reception signal Rs output by each oscillation element 12a based on two or more different reception aperture functions, to obtain the coherence based on the information obtained by performing a cross-correlation operation between the respective reception signals obtained by the weighted delay processing.

In this way, the characteristic estimation unit 20 specifies the signal characteristic of the reception signal for each region of the data space of the reception beam data. FIG. 4 is a conceptual diagram showing reception beam data Rb and a region Re of the reception beam data Rb in the data space. The region Re is defined in advance, and one region Re has a certain width (plurality of pixels in the corresponding ultrasound image). In a case in which the coherence is estimated as the signal characteristic of the reception signal, the characteristic estimation unit 20 sets the coherence estimated based on the individual reception signal Rs before the addition processing as the coherence of each region Re (arranged in the depth direction) corresponding to the reception beam data Rb obtained by performing the reception beam forming of the individual reception signal Rs. It should be noted that which region Re among the regions Re arranged in the depth direction has the coherence estimated based on the individual reception signal Rs can be specified based on an acquisition timing of the amplitude value distribution acquired for specifying the coherence.

### Estimation Method of S/N Ratio

Next, the estimation method of the S/N ratio of the reception signal will be described. The characteristic estimation unit 20 estimates the S/N ratio of the reception signal based on the reception signal (that is, the reception beam data Rb) after the reception beam forming by the transmission/reception unit 14.

With reference to FIG. 4, a signal component S of the S/N ratio is the signal intensity (amplitude) of each region Re of the reception beam data Rb. A noise component N of the S/N ratio can be the signal intensity of each region Re of the reception beam data Rb output from the transmission/reception unit 14 in an environment in which each oscillation element 12a does not receive the reflected waves from the subject (for example, in a case in which the ultrasound waves are transmitted toward air). Therefore, the noise component N of each region Re is acquired in advance, and stored in the memory 32.

In the present embodiment, the characteristic estimation unit 20 first calculates the signal component S of each region Re, not simply the signal intensity (amplitude) of the reception beam data Rb, as follows. The characteristic estimation unit 20 executes frequency analysis processing (for example, fast Fourier transform (FFT)) with respect to the reception beam data Rb before the detection processing in each region Re. As a result, a frequency spectrum of the reception beam data Rb is acquired for each region Re. Next, the characteristic estimation unit 20 calculates a frequency integrated value of the frequency spectrum of each region Re, and sets the calculated frequency integrated value as the signal component S of each region Re.

The characteristic estimation unit 20 estimates the S/N ratio of each region Re based on the signal component S of each region Re calculated as described above and the noise component N of each region Re stored in advance in the memory 32.

### Estimation Method of Attenuation Information

Finally, the estimation method of the attenuation information of the reception signal will be described. The characteristic estimation unit 20 estimates the attenuation information of the reception signal based on the reception signal (reception beam data Rb after the filter processing) after the filter processing by the signal processing unit 16. As described above, the characteristic estimation unit 20 obtains the frequency spectrum by performing the frequency analysis processing on the reception beam data Rb before the detection processing in each region Re, and sets the frequency integrated value of the obtained frequency spectrum as the signal intensity of each region Re. Then, the characteristic estimation unit 20 estimates the attenuation information based on the signal intensity of a plurality of regions Re arranged in the depth direction. For example, the signal intensity of the plurality of regions Re arranged in the depth direction can be plotted in a two-dimensional data space of the depth and the signal intensity, and a slope of an approximate straight line of the signal intensity of each of the plotted regions Re (each depth) can be set as the attenuation information. It should be noted that, in a case in which the characteristic estimation unit 20 specifies the signal characteristic of the reception signal for each region Re of the data space of the reception beam data Rb, the attenuation information of each region Re arranged in the depth direction may be the same as each other.

In a case in which a structure (for example, organ or blood vessel) of the subject is within an irradiation range of the ultrasound waves, the method of attenuating the ultrasound waves considerably differs depending on the structure. Therefore, in the data space of the reception beam data Rb, in a case in which the attenuation information is estimated based on the signal intensity of the region Re corresponding to the structure, appropriate attenuation information cannot be obtained.

Therefore, in the data space of the reception beam data Rb, the characteristic estimation unit 20 estimates the attenuation information by using the signal intensity of the region Re not including the structure, by excluding the signal intensity of the region Re including the structure. The region Re including the structure can be specified based on the reception signal before the detection processing. For example, the characteristic estimation unit 20 can determine whether or not each region Re includes the structure based on a spatial change amount of the frequency component of the reception signal.

### Decision of Image Processing Parameter

In the present embodiment, the image processing parameter decision unit 22 decides, as the image processing parameter, a brightness transformation coefficient (γ coefficient) for transforming the brightness of the pixel of the ultrasound image based on at least one of the coherence or the S/N ratio of the reception signal. In addition, the image processing parameter decision unit 22 decides, as the image processing parameter, a gain applied to each expansion coefficient obtained by multi-resolution analysis of the ultrasound image based on at least one of the coherence or the S/N ratio of the reception signal. Alternatively, the image processing parameter decision unit 22 decides the signal intensity of the reception signal corrected according to the depth of the subject as the image processing parameter based on the attenuation information of the reception signal. Hereinafter, the decision method of each image processing parameter will be described. It should be noted that the image processing parameter decided by the image processing parameter decision unit 22 is not limited to these.

### Decision Method of γ Coefficient

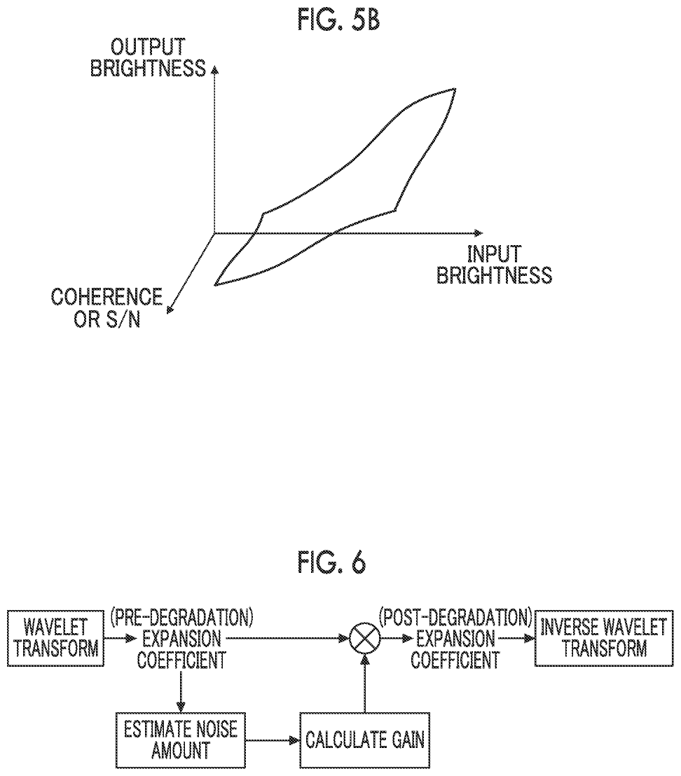

FIG. 5A is a diagram showing an example of a γ curve used in brightness transformation processing. The γ coefficient is a parameter representing the γ curve representing a relationship between the brightness (referred to as an input brightness) of the pixel of the ultrasound image before the brightness transformation processing and the brightness (referred to as an output brightness) of the pixel after the brightness transformation processing.

In a case in which the input brightness is the same, the output brightness is larger as the γ coefficient is larger, and the output brightness is smaller as the γ coefficient is smaller. In the related art, the γ coefficient is a parameter included in the function representing the relationship between the input brightness and the output brightness. However, as shown in FIG. 5A or FIG. 5B, in the present embodiment, the γ coefficient is set as a parameter included in the function representing the relationship between the input brightness and the coherence, and the output brightness. Alternatively, the γ coefficient is set as a parameter included in the function representing the relationship between the input brightness and the S/N ratio, and the output brightness. Alternatively, the γ coefficient may be used as a parameter included in the function representing the relationship between the input brightness, the coherence and the S/N ratio, and the output brightness. The γ coefficient is decided for each region Re.

As described above, as the less noise is included in the reception signal, the coherence of the reception signal is larger. Therefore, in the present embodiment, the image processing parameter decision unit 22 makes the γ coefficient of the region larger as the coherence of the region (hereinafter, referred to as the region Re for the sake of convenience) of the ultrasound image corresponding to each region Re of the reception beam data Rb is larger. As a result, the brightness of the pixel included in the region Re is larger as the coherence is larger. Alternatively, the image processing parameter decision unit 22 makes the γ coefficient of the region larger as the S/N ratio of the region Re is larger. Similarly, as a result, the brightness of the pixel included in the region Re is larger as the S/N ratio is larger. By deciding the γ coefficient in this way, the brightness of the region Re including less noise is larger, while the brightness of the region Re including more noise is smaller, so that the effect of the noise reduction is exhibited.

### Decision Method of Gain Applied to Expansion Coefficient Obtained by Multi-Resolution Analysis

In the present embodiment, a target of the multi-resolution analysis is the ultrasound image formed by the image formation unit 24. As will be described in detail later, the noise of the ultrasound image is reduced by the multi-resolution analysis. The multi-resolution analysis is performed by the image formation unit 24, and the image processing parameter decision unit 22 decides the gain to be applied to the expansion coefficient obtained in the middle of the multi-resolution analysis based on the signal characteristic of the reception signal.

FIG. 6 is an explanatory diagram showing a flow of processing of the multi-resolution analysis. First, the image formation unit 24 performs a wavelet transform with respect to the ultrasound image. In the wavelet transform, a high-pass filter and a low-pass filter are applied to a plurality of edge directions (for example, a horizontal direction (x coordinate direction) and a vertical direction (y coordinate direction) of the ultrasound image) of the ultrasound image, and the ultrasound image is decomposed into signals in each edge direction and at each resolution level. For example, in a case in which the high-pass filter is applied in the horizontal direction of the ultrasound image, an edge component (high-resolution component) in the horizontal direction is extracted, and in a case in which the low-pass filter is applied in the horizontal direction of the ultrasound image, a low-resolution component in the horizontal direction is extracted. Each signal obtained by such decomposition is referred to as an expansion coefficient w. The expansion coefficient w can be represented as in Expression (4).

w=w_{j,o}[m,n] (4)

In Expression (4), j represents a resolution level, and o represents an edge direction. Also, m and n represent positions (coordinates) on the ultrasound image.

The image formation unit 24 estimates the noise amount at the expansion coefficient w for each of a plurality of resolution levels j. Here, since the noise amount at the expansion coefficient w has different characteristic depending on the position (m,n) and the edge direction o, in the present embodiment, the image formation unit 24 calculates, as the noise amount at each resolution level j, a representative value of the plurality of expansion coefficients w at all positions and in all edge directions. For example, in a case in which the noise amount zⱼ of the resolution level j is represented by a standard deviation of the plurality of expansion coefficients w, the noise amount zⱼ is represented by Expression (5).

$${z}_{j} = \frac{{\sum }_{o , m , n} {\vert {w}_{j , 0} [ m , n ] \vert}^{2}}{{N}_{j}} \qquad ( 5 )$$

In Expression (5), Nj is the number of the expansion coefficients w at all positions and in all edge directions at the resolution level j.

Also, for example, in a case in which the noise amount zⱼ of the resolution level j is represented by a median value of an absolute value of the plurality of expansion coefficients w, the noise amount zⱼ is represented by Expression (6).

In Expression (6), α is a constant.

zⱼ=α_{o,m,n}^{Median}|w_{j,o}[m,n]| (6)

Next, the image formation unit 24 applies the gain to each expansion coefficient w. Such gain transformation is referred to as degradation. Basically, a small gain is applied to the expansion coefficient w including a large amount of the noise components, and a large gain is applied to the expansion coefficient w including a large amount of the signal components. The signal component in the ultrasound image has a property of being localized at a specific position and frequency. On the contrary, the noise component has a property of being dispersed at a position and a frequency. For this reason, the absolute value of the expansion coefficient w including a large amount of the signal components tends to be large, in other words, the absolute value of the expansion coefficient w including a large amount of the noise components tends to be small.

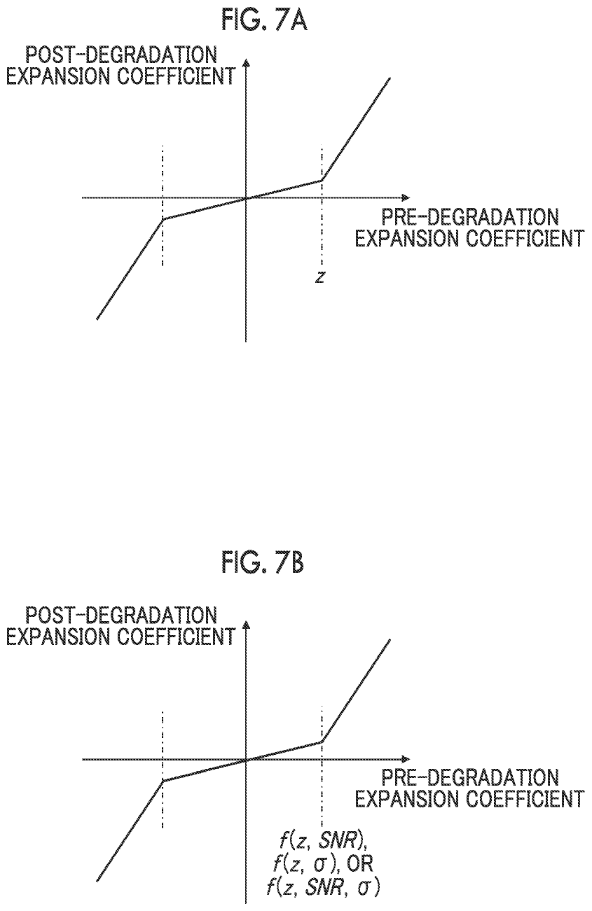

FIG. 7A is a diagram showing an example of an expansion coefficient transformation function in the related art. In the graph shown in FIG. 7A, a horizontal axis represents a pre-degradation expansion coefficient w, and a vertical axis represents a post-degradation expansion coefficient w′. Since there is generally the above-described tendency for the absolute value of the expansion coefficient w and the noise amount z, as shown in FIG. 7A, in the related art, a larger gain is applied to the expansion coefficient w as the absolute value of the expansion coefficient w is larger, and a smaller gain is applied to the expansion coefficient w as the absolute value of the expansion coefficient w is smaller. Further, since the noise amount z for each expansion coefficient w is predicted, a larger gain is applied to the expansion coefficient w as the noise amount z estimated for the expansion coefficient w is smaller, and a smaller gain is applied to the expansion coefficient w as the noise amount z estimated for the expansion coefficient w is larger. For example, as shown in FIG. 7A, a range (mainly, a region in which the absolute value of the pre-degradation expansion coefficient w is small) of the pre-degradation expansion coefficient w to be degraded (gain is small) in the expansion coefficient transformation function is changed according to the noise amount z. As a result, the noise in the ultrasound image is reduced.

As described above, in the related art, the expansion coefficient transformation function is a function of the expansion coefficient w and the noise amount z. The post-degradation expansion coefficient w′ in the related art can be represented, for example, by Expression (7).

|w′_{j,o}[m,n]|=A(w_{j,o}[m,n];z_{j,o}[m,n]) (7)

In Expression (7), A(p;z) is an amplitude transformation function, and is a function that is monotonically increased with respect to the input p.

In the present embodiment, the image processing parameter decision unit 22 corrects the gain according to the magnitude of the absolute value of each expansion coefficient w described above to be further larger as at least one of the coherence of the reception signal or the S/N ratio of the reception signal, which is estimated by the characteristic estimation unit 20, is larger. In other words, in the present embodiment, as shown in FIG. 7B, the expansion coefficient transformation function representing the gain for each expansion coefficient w is a function of the expansion coefficient w, the noise amount z, and at least one of the coherence or the S/N ratio of the reception signal.

For example, as shown in FIG. 7B, a range (mainly, a region in which the absolute value of the pre-degradation expansion coefficient w is small) of the pre-degradation expansion coefficient w to be degraded (gain is small) in the expansion coefficient transformation function is held functionally to be changed according to the coherence or the S/N ratio. Alternatively, the expansion coefficient transformation function may be held as a multi-dimensional function in which an axis of coherence or the S/N ratio is added, not a one-dimensional function for transforming the pre-degradation expansion coefficient w into the post-degradation expansion coefficient w′ as shown in FIG. 7A.

In a case in which the expansion coefficient transformation function is a function of the expansion coefficient w, the noise amount z, and the coherence, the post-degradation expansion coefficient w′ can be represented by, for example, Expression (8).

|w′_{j,o}[m,n]|A(w_{j,o}[m,n];f(z_{j,o}[m,n],a[m,n])) (8)

In Expression (8), 6[m,n] represents the coherence (at the coordinates (m,n) in the region Re) in each region Re.

Further, in a case in which the expansion coefficient transformation function is a function of the expansion coefficient w, the noise amount z, and the S/N ratio, the post-degradation expansion coefficient w′ can be represented by, for example, Expression (9).

|w′_{j,o}[m,n]|=A(w_{j,o}[m,n];f(z_{j,o}[m,n],SNR[m,n])) (9)

In Expression (9), SNR[m,n] represents the S/N ratio (at the coordinates (m,n) in the region Re) in each region Re.

Further, in a case in which the expansion coefficient transformation function is a function of the expansion coefficient w, the noise amount z, the coherence, and the S/N ratio, the post-degradation expansion coefficient w′ can be represented by, for example, Expression (10).

|w′_{j,o}[m,n]|=A(w_{j,o}[m,n];f(z_{j,o}[m,n],a[m,n],SNR[m,n])) (10)

The image formation unit 24 applies the gain decided by the image processing parameter decision unit 22 to each pre-degradation expansion coefficient w to obtain a plurality of post-degradation expansion coefficients w′. Thereafter, as shown in FIG. 6, the image formation unit 24 performs an inverse wavelet transform with respect to the plurality of post-degradation expansion coefficients w′ to obtain the ultrasound image in which the noise is reduced.

As described above, the less noise is included as the coherence or the S/N ratio of the reception signal is larger, in other words, the more noise is included as the coherence or the S/N ratio of the reception signal is smaller. In the expansion coefficient transformation function in the present embodiment, a larger gain is applied to each expansion coefficient w as at least one of the coherence or the S/N ratio of the reception signal is larger, in other words, a smaller gain is applied as at least one of the coherence or the S/N ratio of the reception signal is smaller. As a result, the effect of the noise reduction by the multi-resolution analysis is increased.

### Decision Method of Brightness Correction Function for Time Gain Control

The image processing parameter decision unit 22 decides a brightness correction function for time gain control (TGC) as the image processing parameter based on the attenuation information estimated by the characteristic estimation unit 20. The brightness correction function is a function representing a relationship between the depth of the subject and the gain (degree of amplification of the signal intensity of the reception beam data Rb). The brightness correction function has a larger gain as the depth of the subject is larger. In the present embodiment, as described above, since the characteristic estimation unit 20 specifies the attenuation information by excluding the region Re including the structure of the subject, even in a case in which there is data corresponding to the structure of the subject in the data space of the reception beam data Rb, the brightness correction function that is not affected by the structure can be decided.

As described above, the attenuation information is decided for each row of the regions Re arranged in the depth direction. However, it is not appropriate to perform the TGC with the brightness correction functions different from each other for each region Re arranged in the depth direction. Therefore, the image processing parameter decision unit 22 decides the entire brightness correction function corresponding to one ultrasound image based on a plurality of brightness correction functions corresponding to the rows of the plurality of regions Re. For example, the entire brightness correction function is decided by averaging the plurality of brightness correction functions.

### Image Quality Enhancement Processing Based on Decided Image Processing Parameter

The image formation unit 24 executes the image quality enhancement processing of enhancing an image quality of the ultrasound image based on the image processing parameter decided by the image processing parameter decision unit 22.

In a case in which the γ coefficient is decided as the image processing parameter, the image formation unit 24 transforms the brightness for each region Re of the formed ultrasound image based on the γ curve represented by the γ coefficient decided for each region Re. As a result, the ultrasound image in which the noise is reduced is formed.

In a case in which the gain to be applied to the expansion coefficient obtained by the multi-resolution analysis is decided as the image processing parameter, as described above, the image formation unit 24 forms the ultrasound image in which the noise is reduced by the multi-resolution analysis using the decided gain.

In a case in which a brightness correction function (overall brightness correction function) for TGC is decided as the image processing parameter, the image formation unit 24 executes TGC on the ultrasound image based on the overall brightness correction function. In the present embodiment, even in the case in which the structure of the subject is included in the ultrasound image, the image formation unit 24 executes the TGC with respect to the region of the ultrasound image including the structure. As a result, the ultrasound image in which the brightness in the depth direction is suitably corrected is formed.

### Effects of Ultrasound Diagnostic Apparatus According to Present Embodiment

The outline of the ultrasound diagnostic apparatus 10 according to the present embodiment is described above. In the ultrasound diagnostic apparatus 10, the reception signal before the detection processing, which has an abundant amount of information, is analyzed to estimate the signal characteristic of the reception signal, and the image quality enhancement processing of the ultrasound image is executed based on the image processing parameter decided based on the estimated signal characteristic. As a result, as compared with the related art, particularly, as compared with the image quality enhancement processing based on the signal characteristic of the reception signal obtained by analyzing the reception signal after the detection processing, a more suitable image quality enhancement processing (processing of realizing lower noise or more suitable TGC) can be performed.

For example, in the embodiment described above, the characteristic estimation unit 20 estimates the coherence as the signal characteristic of the reception signal. Since the phase information of the reception signal is lost by the detection processing, the coherence cannot be estimated from the reception signal after the detection processing. The coherence can be estimated only after analyzing the reception signal before the detection processing, and the image quality enhancement processing can be performed with the estimated coherence.

In addition, in the embodiment described above, the characteristic estimation unit 20 calculates the frequency spectrum of the reception beam data Rb for each region Re having a certain width, and estimates the S/N ratio or the attenuation amount of the ultrasound image based on the frequency spectra of the plurality of regions Re arranged in the depth direction. By performing the image quality enhancement processing based on the image processing parameter decided from the attenuation amount estimated in this way, at least, as compared with a case in which the image quality enhancement processing is performed based on the analysis with respect to the ultrasound image (reception signal after the detection processing), an influence of a speckle (image of a stripe pattern generated in the ultrasound image due to the interference between the scattered waves generated at an unspecified larger number of places in the subject with each other) can be reduced.

### Flow of Processing of Ultrasound Diagnostic Apparatus

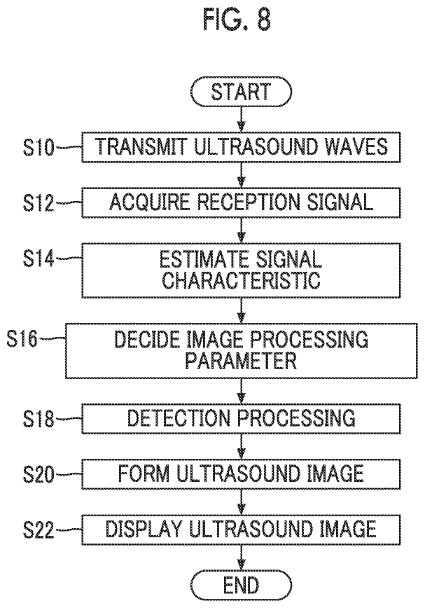

Hereinafter, a flow of the processing of the ultrasound diagnostic apparatus 10 will be described with reference to the flowchart shown in FIG. 8.

In step S10, the transmission/reception unit 14 supplies the transmission signal to the ultrasound probe 12. As a result, the ultrasound waves are transmitted from the plurality of oscillation elements 12a of the ultrasound probe 12 to the subject.

In step S12, the plurality of oscillation elements 12a of the ultrasound probe 12 receive the reflected waves from the subject and transmit the reception signal to the transmission/reception unit 14. As a result, the transmission/reception unit 14 acquires the reception signal. The transmission/reception unit 14 transmits the reception signal before the detection processing (individual reception signal Rs before the addition processing by the transmission/reception unit 14, the reception signal (reception beam data Rb) after the addition processing by the transmission/reception unit 14, or the reception beam data Rb after the filter processing in the signal processing unit 16) to the characteristic estimation unit 20.

In step S14, the characteristic estimation unit 20 analyzes the reception signal before the detection processing. As a result, the signal characteristic of the reception signal is estimated.

In step S16, the image processing parameter decision unit 22 decides the image processing parameter for enhancing an image quality of the ultrasound image based on the signal characteristic of the reception signal estimated in step S14.

In step S18, the detection processing unit 18 executes the detection processing with respect to the reception beam data Rb from the signal processing unit 16. It should be noted that the processing of steps S14 and S16 and step S18 can be executed in parallel.

In step S20, the image formation unit 24 forms the ultrasound image (B-mode image) based on the reception beam data Rb after the detection processing. In addition, the image formation unit 24 executes, in the formation processing of the ultrasound image or with respect to the formed ultrasound image, image quality enhancement processing of the ultrasound image using the image processing parameter decided in step S16.

In step S22, the display controller 26 displays the ultrasound image formed in step S20 in which the image quality is enhanced, on the display 28.

Although the embodiment according to the present invention has been described above, the present invention is not limited to the embodiment described above, and various modifications can be made without departing from the gist of the present invention.

For example, in the present embodiment, the ultrasound probe 12 is the probe including the oscillation elements arranged in a row, but the ultrasound probe 12 may be a two-dimension (2D) array probe including oscillation elements arranged in two dimensions. The reception signal, which is the processing target of each of the units of the ultrasound diagnostic apparatus 10, may constitute three-dimensional volume data obtained by the 2D array probe and extending in the depth direction, an azimuth direction, and a slice direction.

What is claimed is:

1. An ultrasound diagnostic apparatus, comprising:
a characteristic estimation unit that analyzes a reception signal before detection processing among reception signals obtained by transmitting and receiving ultrasound waves with respect to a subject, to estimate a signal characteristic of the reception signal for each region in a data space of the reception signal;
an image processing parameter decision unit that decides an image processing parameter based on the estimated signal characteristic for each region; and
an image formation unit that forms an ultrasound image subjected to image quality enhancement processing based on the image processing parameter based on the reception signal subjected to the detection processing.

2. The ultrasound diagnostic apparatus according to claim 1,
wherein the signal characteristic is at least one of coherence of each reception signal output by each oscillation element that transmits and receives the ultrasound waves, an S/N ratio calculated by frequency analysis with respect to the reception signal, or attenuation information indicating attenuation of signal intensity in a depth direction of the subject, which is calculated by the frequency analysis with respect to the reception signal, the attenuation information being estimated by excluding the signal intensity of the region which includes a structure in the subject and is specified based on the reception signal before the detection processing.

3. The ultrasound diagnostic apparatus according to claim 2,
wherein the image processing parameter is a brightness transformation coefficient for transforming brightness of a pixel of the ultrasound image based on at least one of the coherence or the S/N ratio related to the region, and
the brightness transformation coefficient is a coefficient for making brightness of a pixel included in the region larger as at least one of the coherence or the S/N ratio related to the region is larger.

4. The ultrasound diagnostic apparatus according to claim 2,
wherein the image formation unit performs multi-resolution analysis with respect to the ultrasound image formed based on the reception signal subjected to the detection processing and executes noise reduction of the ultrasound image by applying a gain according to magnitude of an absolute value of each obtained expansion coefficient to each expansion coefficient, and
the image processing parameter is the gain corrected to be larger as at least one of the coherence or the S/N ratio related to the region is larger.

5. The ultrasound diagnostic apparatus according to claim 2,
wherein the image processing parameter is a brightness correction function for time gain control based on the attenuation information, and
the image formation unit executes the time gain control with respect to the region of the ultrasound image including the structure based on the brightness correction function.
